(19) United States
(12) Patent Application Publication
(10) Pub. No.: US 2009/0227866 A1
(43) Pub. Date: Sep. 10, 2009

(54) ULTRASOUND ENDOSCOPIC INTRALUMINAL ORGAN TREATMENT METHOD

(75) Inventors: Takeharu NAKAZATO, Tokyo (JP); Shinichi TSUTAKI, Tokyo (JP); Sunao SATO, Yamato-shi (JP); Akiko MIZUNUMA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/042,731

(22) Filed: Mar. 5, 2008

(51) Int. Cl. A61B 1/313; A61B 8/13

(52) U.S. Cl. 600/439; 600/104; 128/898

(57) ABSTRACT

In an ultrasound endoscopic intraluminal organ treatment method according to the present invention, a surgeon forms a first opening in a wall of a first luminal organ while observing an optical image and an ultrasound image formed by an ultrasound endoscope led into the first luminal organ through a natural orifice, pulls a part of a desired second luminal organ adjacent to the first luminal organ into the inside of the first luminal organ from the first opening through an abdominal cavity, and applies treatment to the second luminal organ in the inside of the first luminal organ.

### BACKGROUND OF THE INVENTION

1. Field of the Invention

The present invention relates to an ultrasound endoscopic intraluminal organ treatment method, and, more particularly to an ultrasound endoscopic intraluminal organ treatment method for carrying out treatment by an ultrasound endoscope in luminal organs arranged through natural orifices in a body cavity.

2. Description of the Related Art

In recent years, researches concerning surgery called NOTES™ surgery (Natural Orifice Translumenal Endoscopic Surgery) for forming, while observing an image formed by an endoscope led into a luminal organ through a natural orifice, an opening on a wall surface of the luminal organ, leading the endoscope into an abdominal cavity from the opening, and performing observation and treatment for intra-abdominal organs have been actively performed. Various proposals concerning endoscopes and treatment instrument systems used for the surgery have been made by, for example, Japanese Patent Laid-Open No. 2004-267772.

On the other hand, it is disclosed that an opening or the like is temporarily held by using a T-bar unit such as “T-ANCHOR SUTURING DEVICE” described in, for example, U.S. Pat. No. 5,626,614 and a technique for making it possible to efficiently carrying out manipulation has been developed.

### SUMMARY OF THE INVENTION

An ultrasound endoscopic intraluminal organ treatment method according to claim 1 of the present invention includes:

an endoscope inserting step of inserting an insertion portion of an ultrasound endoscope including one or more treatment instrument channels into a first object luminal organ from a natural orifice of a patient, the insertion portion having at a distal end thereof an optical observation portion for optically observing an observation region and an ultrasound observation portion that is capable of observing the observation region with ultrasound;

an adjacent organ confirming step of confirming a second object luminal organ adjacent to an outer wall surface of the first object luminal organ under an ultrasound observation of the ultrasound endoscope;

a capturing step of capturing the second object luminal organ under the ultrasound observation of the ultrasound endoscope;

an opening portion forming step of forming an opening portion in a luminal wall of the first object luminal organ;

a pulling-in step of pulling a part of the captured second object luminal organ into the inside of the first object luminal organ from the opening portion; and

a treatment step of applying treatment to the part of the second object luminal organ pulled into the inside of the first object luminal organ.

Other characteristics and advantages of the present invention will be sufficiently made apparent through the following explanation.

### BRIEF DESCRIPTION OF THE DRAWINGS

FIGS. 1 to 5 relate to a first embodiment of the present invention. FIG. 1 is a schematic diagram showing the entire structure of an ultrasound endoscope system. FIG. 2 is an external view showing the structure of a distal end of the ultrasound endoscope shown in FIG. 1. FIG. 3 is a diagram showing the structure of a T-bar portion of a T-bar unit inserted through the ultrasound endoscope shown in FIG. 1 and used. FIG. 4 is a diagram showing the structure of a T-bar injection portion of the T-bar unit inserted through the ultrasound endoscope shown in FIG. 1 and used. FIG. 5 is a diagram showing the structure of a guide wire with stopper inserted through a hollow portion of a piercing treatment instrument used via treatment instrument channels of the ultrasound endoscope shown in FIG. 1.

FIGS. 6 to 17 relate to the first embodiment of the present invention. FIG. 6 is an overall diagram showing a system arrangement configuration for explaining actions of the ultrasound endoscope system shown in FIG. 1. FIG. 7 is a flowchart for explaining a flow of intraluminal organ treatment method by an ultrasound endoscope shown in FIG. 6. FIG. 8 is a first diagram for explaining the flowchart of FIG. 7. FIG. 9 is a second diagram for explaining the flowchart of FIG. 7. FIG. 10 is a third diagram for explaining the flowchart of FIG. 7. FIG. 11 is a fourth diagram for explaining the flowchart of FIG. 7. FIG. 12 is a fifth diagram for explaining the flowchart of FIG. 7. FIG. 13 is a sixth diagram for explaining the flowchart of FIG. 7. FIG. 14 is a seventh diagram for explaining the flowchart of FIG. 7. FIG. 15 is an eighth diagram for explaining the flowchart of FIG. 7. FIG. 16 is a ninth diagram for explaining the flowchart of FIG. 7. FIG. 17 is a tenth diagram for explaining the flowchart of FIG. 7.

FIGS. 18 to 21 relate to a modification of the first embodiment of the present invention. FIG. 18 is a flowchart for explaining a modification of the processing shown in FIG. 7. FIG. 19 is a first diagram for explaining an action in the flowchart of FIG. 18. FIG. 20 is a second diagram for explaining the action in the flowchart of FIG. 18. FIG. 21 is a third diagram for explaining the action in the flowchart of FIG. 18.

### DETAILED DESCRIPTION OF THE PREFERRED EMBODIMENTS

The present invention will be hereinafter explained with reference to embodiments shown in the figures.

### First Embodiment

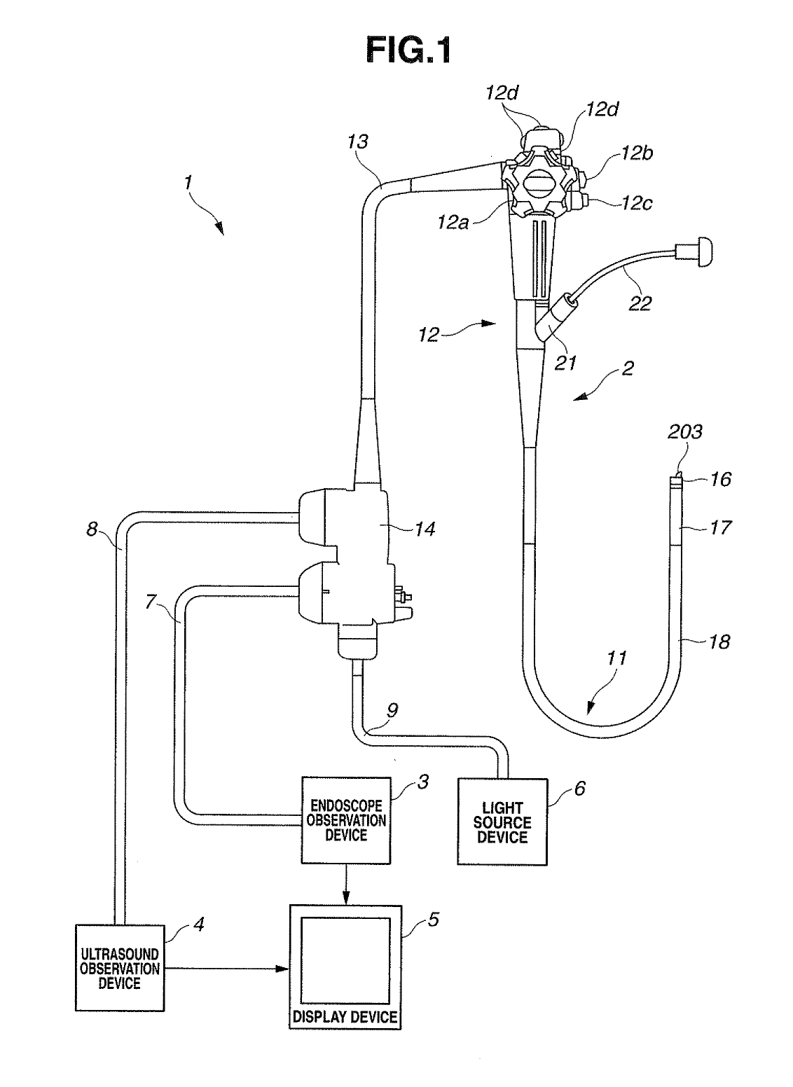

As shown in FIG. 1, an ultrasound endoscope system 1 according to the present embodiment mainly includes an ultrasound endoscope 2 that includes an endoscope observing unit having an endoscope observing function and an ultrasound observation unit having an ultrasound observation function, an endoscope observation device 3 that processes control of the endoscope observing unit and an output signal thereof, an ultrasound observation device 4 that processes control of the ultrasound observation unit and an output signal thereof, a display device 5 that receives the respective signals from the endoscope observation device 3 and the ultrasound observation device 4 and appropriately displays an endoscope image and an ultrasound tomographic image, a light source device 6 that is a light source of illumination light emitted from a front surface of a distal end of the endoscope observing unit, a video cable 7, an ultrasound cable 8, and a light source cable 9.

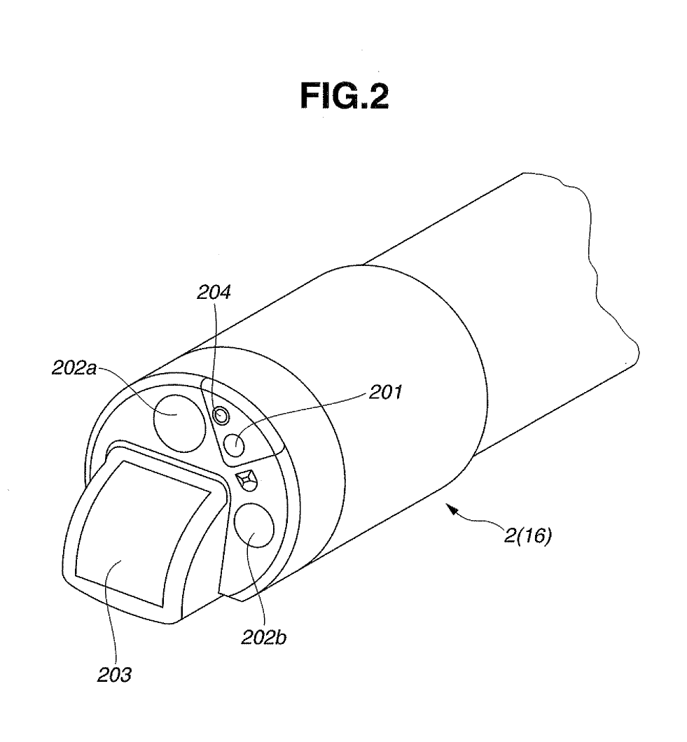

The ultrasound endoscope 2 in the ultrasound endoscope system 1 is, as shown in FIG. 2, a convex-type ultrasound endoscope that has at a distal end thereof an ultrasound transducer unit 203 for scanning, for example, a surface parallel to a front side of an endoscope insertion axis. A direct-vision convex-type ultrasound endoscope device or the like is applied as the ultrasound endoscope 2. In the ultrasound endoscope 2, two treatment instrument channels (not shown) are arranged such that, on a distal end surface, an observation direction of an observation window 201 for guiding an observation image to an image pickup device (not shown) in the distal end and a direction in which distal end opening portions 202a and 202b of the treatment instrument channels (not shown), which are inserted through the insertion portion 11 of the ultrasound endoscope 2, face are substantially parallel to the endoscope insertion axis. The distal end opening portion 202a is arranged to be placed on an ultrasound scanning surface.

In the distal end surface of the ultrasound endoscope 2, an illumination window 204 for emitting illumination light from the light source device 6 is provided adjacent to the observation window 201. The illumination light emitted from the illumination window 204 is transmitted through an optical fiber bundle (not shown) inserted through the insertion portion 11 from the light source device 6.

Referring back to FIG. 1, the ultrasound endoscope 2 mainly includes the slim insertion portion 11 inserted into a body cavity, an operation portion 12 continuously provided on a proximal end side of the insertion portion 11 and used for operating the insertion portion 11, a universal cable 13 extending from a side of the operation portion 12, and a connector portion 14 provided at one end of the universal cable 13.

The insertion portion 11 mainly includes, in order from a distal end side thereof, a distal end rigid portion 16 formed of a rigid member, a bending portion 17 continuously provided on a proximal end side of the distal end rigid portion 16 and formed to freely bend, and a flexible tube portion 18, one end of which is continuously provided on a proximal end side of the bending portion 17 and the other end of which is continuously provided on a distal end side of the operation portion 12, and that is formed thin and long with flexibility. In the insertion portion 11, plural, for example, two treatment instrument channels (not specifically shown in the figure) inserted through between the proximal end and the distal end thereof are formed.

In the inside of the distal end side of the distal end rigid portion 16, although not shown in the figure, an endoscope observing unit and an ultrasound observation unit are disposed. In the ultrasound observation unit, plural ultrasound transducers that transmit and receive ultrasounds are arrayed to form an ultrasound scanning surface. The ultrasound observation unit can acquire an ultrasound signal that contributes to creation of a tomographic image further in the inside than a body cavity wall (an ultrasound tomographic image). The endoscope observing unit has an observation optical member, an illumination optical member, an image pickup device, and the like. The endoscope observing unit can obtain an image pickup signal that contributes to generation of an image signal for optically picking up an image of the surface of the body cavity wall and displaying an endoscope image for observation.

The operation portion 12 includes operation members for performing various kinds of operation of the ultrasound endoscope 2 such as an angle knob 12a that is an operation member for freely carrying out bending operation on the bending portion 17 of the insertion portion 11 in up, down, left and right directions, a suction button 12b for performing suction operation, an air-supply and water-supply button 12c for performing air supply and water supply operation, and plural operation members 12d for performing various kinds of operation such as display switching for the display device 5 and freeze instruction and release instruction for a display image.

Further, in the operation portion 12, plural treatment instrument insertion openings 21 (only one treatment instrument insertion opening is shown in the figure) serving as insertion openings for inserting, in using the ultrasound endoscope 2, the ultrasound endoscope 2 through treatment instrument channels (not shown) in the insertion portion 11 and leading various treatment instruments 22 into the body cavity are provided near the distal end side in a state projecting from the operation portion 12.

The universal cable 13 is a cable that is disposed to extend from the side of the operation portion 12 as described above and through which various plural signal lines and the like for transmitting electric signals and the like and an optical fiber cable bundle and the like for illumination light are inserted. A connector portion 14 for securing connection of the respective devices of the endoscope observation device 3, the ultrasound observation device 4, and the light source device 6 to the ultrasound endoscope 2 are disposed at a distal end portion of the universal cable 13.

The endoscope observation device 3 is an optical image processing device that controls to drive the image pickup device of the endoscope observing unit of the ultrasound endoscope 2 to receive an image pickup signal transmitted from the image pickup device, perform various kinds of signal processing, and generate a video signal for an endoscope optical observation image.

The ultrasound observation device 4 is an ultrasound image processing device that controls to drive an ultrasound transducer of an ultrasound transducer unit 203 of the ultrasound endoscope 2 to transmit ultrasound of a predetermined frequency to an observation object and receive, from the ultrasound transducer, an electric signal obtained by receiving the ultrasound reflected by the observation object. The ultrasound observation device 4 performs various kinds of signal processing to thereby generate a video signal for an ultrasound tomographic image.

The display device 5 is a device that receives the various video signals generated by the ultrasound observation device 4 and the endoscope observation device 3 to display observation images corresponding to the video signals, respectively, i.e., the ultrasound tomographic image and the optical observation image by appropriately switching the images or simultaneously display the images.

The light source device 6 is a device that supplies illumination light for illuminating the front of the ultrasound endoscope 2 through an illumination window 204 provided on a front portion at the distal end of the endoscope observing unit of the ultrasound endoscope 2.

The ultrasound cable 8 is a connection cable that electrically connects the ultrasound observation device 4 and the ultrasound endoscope 2.

The video cable 7 is a connection cable that electrically connects the endoscope observation device 3 and the ultrasound endoscope 2.

The light source cable 9 is an optical fiber cable made of an optical fiber bundle that connects the light source device 6 and the ultrasound endoscope 2 and guides the illumination light from the light source device 6 to the illumination window 204 of the endoscope observing unit in the ultrasound endoscope 2.

The plural (two in the present embodiment) treatment instrument insertion openings 21 (only one treatment instrument insertion opening is shown in the figure) provided in the operation portion 12 of the ultrasound endoscope 2 communicate with the treatment instrument channels (not shown) formed to be inserted through to the distal end opening portions 202a and 202b (see FIG. 2) provided on the front side of the distal end rigid portion 16 from the inside of the operation portion 12 via the inside of the insertion portion 11. Therefore, the various treatment instruments 22 inserted from the treatment instrument insertion openings 21 can be inserted through the treatment instrument channels and projected from and retracted into the distal end opening portions 202a and 202b of the insertion portion 11.

Examples of the treatment instruments 22 inserted from the plural treatment instrument insertion openings 21 include a high-frequency treatment instrument such as a needle knife as an opening forming treatment instrument that is a surgical treatment instrument (an opening treatment instrument for an endoscope) that is used for dissection of a desired region of the luminal organs to form an opening portion under observation by the ultrasound endoscope 2, a balloon treatment instrument as an expansion treatment instrument for expanding the opening portion, and a T-bar unit as a holding treatment instrument that performs treatment for holding an opening state of the opening portion.

In the present embodiment, as described above, the surgical treatment instrument 22 for performing a surgical operation of the intra-abdominal organs under the observation by the ultrasound endoscope 2, a suturing treatment instrument for suturing the opening portion opened under the observation by the ultrasound endoscope 2, and the like are inserted into the treatment instrument channels from the plural treatment instrument insertion openings 21. This makes it possible to perform various kinds of treatment such as the surgical operation of the intra-abdominal organs under the observation by the ultrasound endoscope 2.

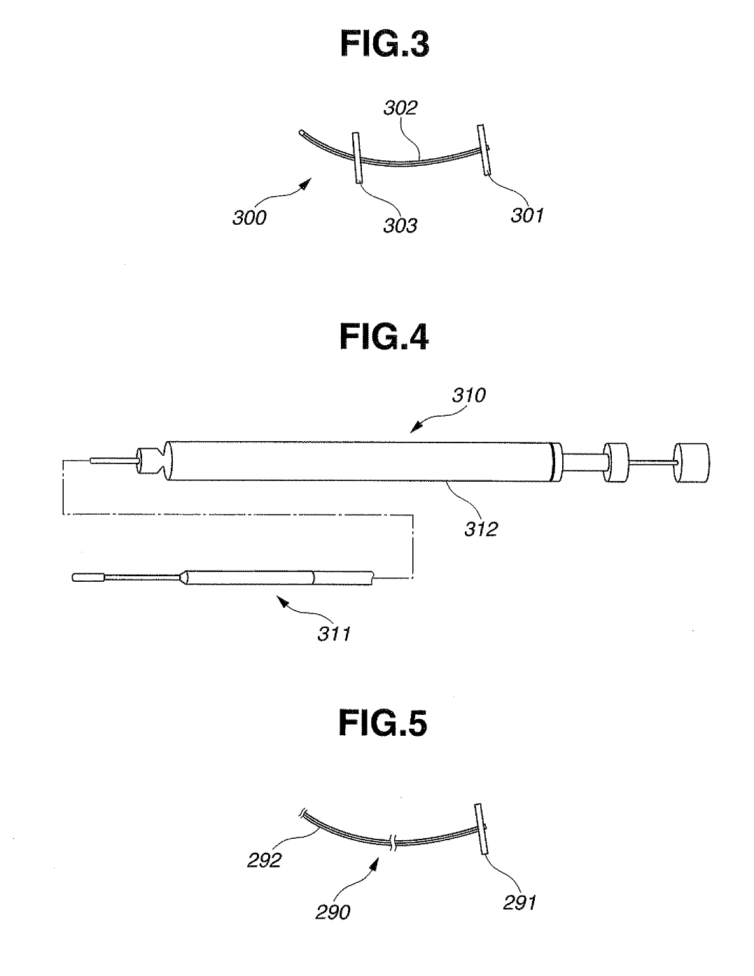

The T-bar unit includes a T-bar portion 300 shown in FIG. 3 and a T-bar injection portion 310 shown in FIG. 4. The T-bar portion 300 includes a bar-like distal end bar member 301, a thread-like member 302 that is connected to the center of the distal end bar member 301 and has the distal end bar member 301 at a distal end thereof, and a center bar member 303, to the center of which the thread-like member 302 is connected. By moving the center bar member 303 along the thread-like member 302, it is possible to hold an organism tissue with the distal end bar member 301 and the center bar member 303. The T-bar injection portion 310 includes a needle-like member 311 having a hollow path through which the T-bar portion 300 is inserted and a T-bar push-pull portion 312 (e.g., equivalent to a syringe of an injector) that can push out the T-bar portion 300 in the hollow path of the needle-like member 311 to a distal end side of the needle-like member 311. The needle-like member 311 can be inserted through the treatment instrument channels.

In the T-bar injection portion 310, a guide wire 290 having a stopper 291 at a distal end thereof (hereinafter referred to as G. W. with stopper) shown in FIG. 5 can be inserted through the hollow path of the needle-like member 311. Like the T-bar portion 300, the G. W. with stopper 290 in the hollow path of the needle-like member 311 can be pushed out to the distal end side. The length of a thread-like wire portion 292 of the G. W. with stopper 290 is sufficiently longer than the length of the insertion portion 11 such that, even in a state in which the stopper 291 is retained in the front at the distal end of the insertion portion 11, predetermined length from the treatment instrument insertion openings 21 remains at a proximal end of the wire portion 292.

An example of manipulation in the prior art same as that in the present embodiment is explained. For example, a bypass method such as gastrojejunostomy has been conventionally carried out by celiotomy or under a laparoscope in the past. However, since invasiveness against a patient is high, less invasive treatment is demanded.

Therefore, as an example of low invasive manipulation in the prior art, for example, there are those disclosed in documents of (1) to (3) described below.
(1) GASTROINTESTINAL ENDOSCOPY: 2005, Volume 62, No. 2, P. 287 to P. 292
(2) GASTROINTESTINAL ENDOSCOPY: 2006, Volume 63, No. 2, P. 307 to P. 312
(3) GASTROINTESTINAL ENDOSCOPY: 2006, Volume 63, No. 2, P. 302 to P. 306

The document (1) discloses manipulation for performing treatment by taking out an endoscope inserted into a luminal organ through a natural orifice to the outside of a lumen from the luminal organ. However, since such manipulation is manipulation performed in an abdominal cavity outside the lumen, it is necessary to secure, for example, a space for performing the manipulation. There are problems in which the treatment is complicated and in which likelihood of causing complication is nonnegligible because surgical treatment is performed in the abdominal cavity outside the lumen.

The document (2) discloses manipulation for pulling a second luminal organ into a first luminal organ and dissecting and suturing the second luminal organ. However, in the dissecting and suturing manipulation, means for checking a projecting state of a treatment instrument for performing treatment is neither disclosed nor indicated. Therefore, there is a problem in that a risk to other organs during the treatment is high.

The document (3) discloses manipulation for pulling a lymph node into a lumen and endoscopically excising the lymph node. However, in the lymph node excising manipulation, a lymph node is identified under an ultrasound observation using an ultrasound endoscope having one treatment instrument channel, means for pulling the lymph node into a lumen is retained, and, thereafter, the ultrasound endoscope is removed and replaced with an optical endoscope having plural channels, and the remaining treatment is carried out by the optical endoscope. In such manipulation, for example, when an ultrasound observation is necessary during the manipulation, endoscopes have to be frequently replaced, for example, it is necessary to replace the optical endoscope with the ultrasound endoscope. Therefore, there is a problem in that the manipulation is not only complicated but also takes time to deteriorate manipulation efficiency.

Therefore, it is an object of an ultrasound endoscopic intraluminal organ treatment method according to the present embodiment to, in order to solve these problems, make it possible to carry out less invasive, safer, and more efficient endoscope treatment while checking regions around a lumen under an optical observation and an ultrasound observation.

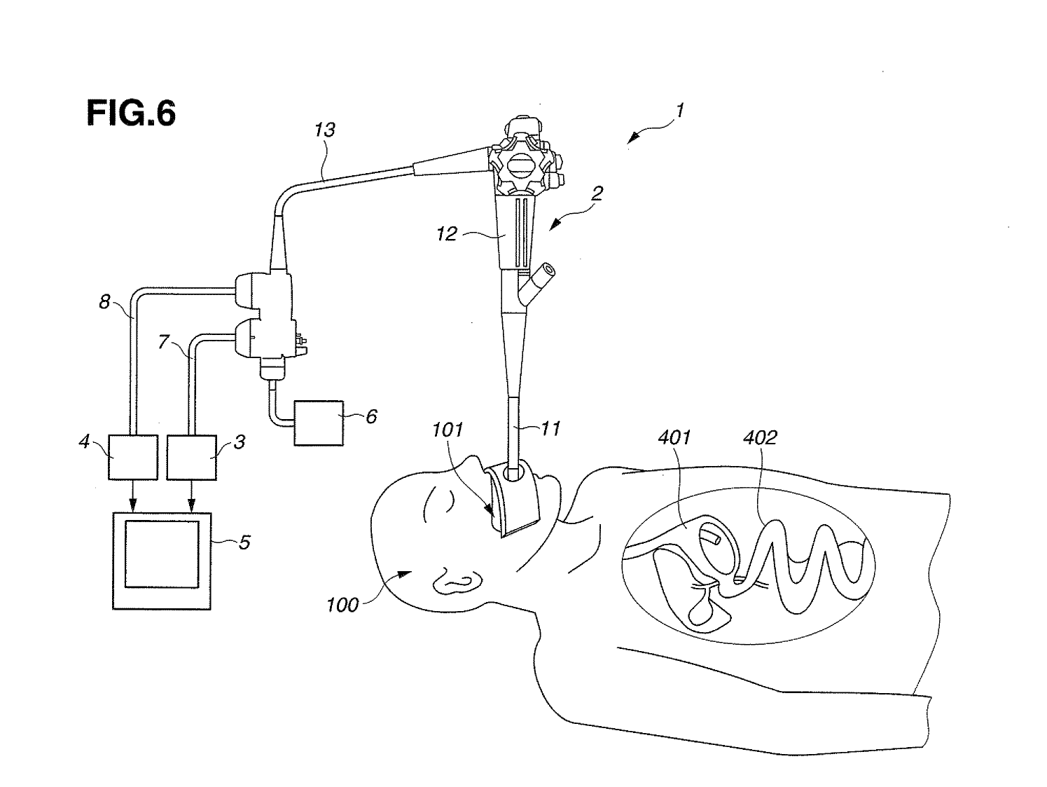

Manipulation performed by using the ultrasound endoscope system 1 according to the present embodiment is explained below with reference to FIGS. 6 to 17. FIGS. 6 to 17 are diagrams for explaining a procedure and the like of the manipulation and conceptually show, for example, an arrangement relation of various organs and the like in the body cavity.

Endoscope manipulation performed by using the ultrasound endoscope system 1 according to the present embodiment explained below is a kind of an ultrasound endoscopic intraluminal organ treatment method for observing and treating luminal organs of a patient.

In the ultrasound endoscopic intraluminal organ treatment method according to the present embodiment, conceptually, a surgeon forms an opening portion in a wall of a first luminal organ while observing optical and ultrasound images formed by an ultrasound endoscope inserted into the first luminal organ through a natural orifice. The surgeon pulls a part of a desired second luminal organ, which is adjacent to the first luminal organ, from the opening portion into the inside of the first luminal organ through an abdominal cavity and applies treatment to the second luminal organ in the inside of the first luminal organ.

First, in FIG. 6, the surgeon energizes the various devices in the ultrasound endoscope system 1 to bring the system into a usable state.

In the state, the surgeon inserts the insertion portion 11 of the ultrasound endoscope 2 into, for example, the inside of a stomach 401, which is a first luminal organ in a tube wall of which an opening is formed, from a natural orifice, for example, a mouth cavity 101 of a patient 100 or the like while observing an optically-acquired endoscope image displayed on the display device 5. Here, the first luminal organ refers to an organ into which an (ultrasound) endoscope can be inserted from the natural orifice in a normal procedure. Therefore, operation for inserting the ultrasound endoscope 2 according to the present embodiment up to this stage is operation same as a flexible endoscope test normally carried out in general.

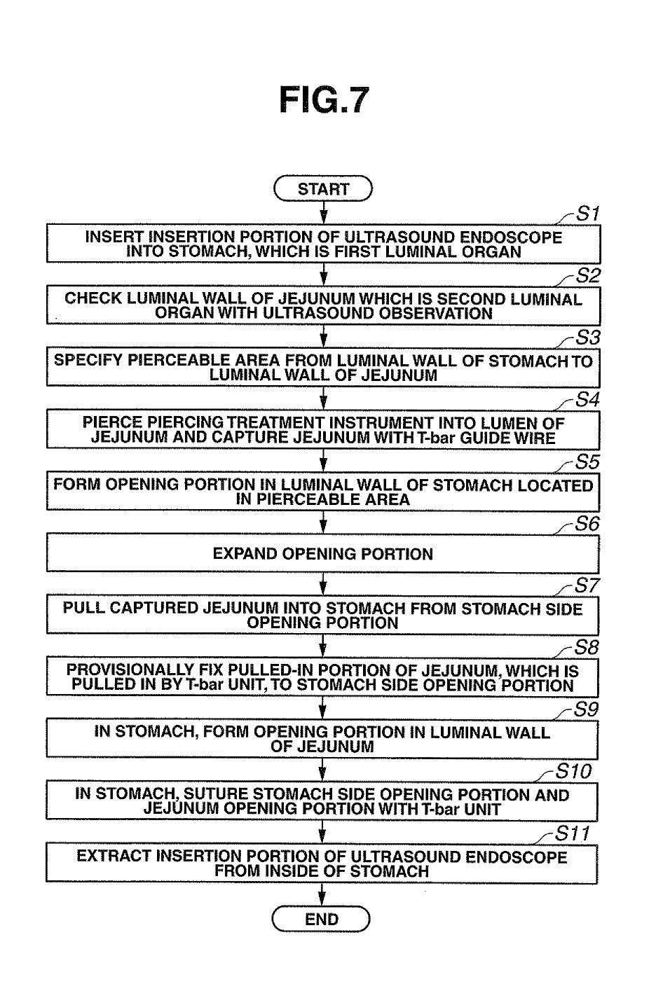

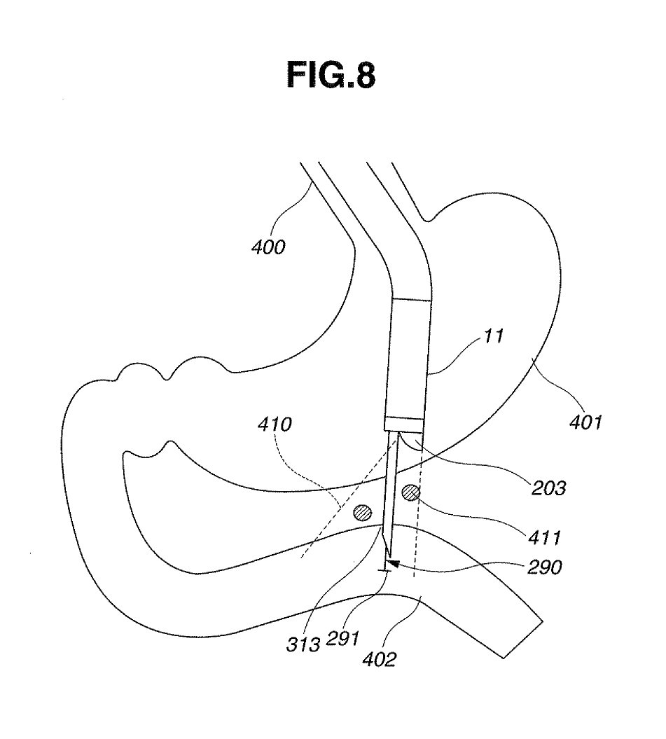

Specifically, using the ultrasound endoscope system 1, the surgeon starts, as shown in FIG. 7, as the endoscope inserting step, in step S1, insertion of the insertion portion 11 of the ultrasound endoscope 2 into the natural orifice, for example, the mouth cavity 101 of the patient 100 or the like (see FIG. 6). Using the ultrasound endoscope system 1, the surgeon checks, with an optical observation of the ultrasound endoscope 2, the insertion of the insertion portion 11 into, for example, the stomach 401, which is the first object luminal organ in the tube wall of which an opening is formed, from an esophagus 400 (see FIG. 8).

Next, using the ultrasound endoscope system 1, the surgeon sets, as an adjacent organ confirming step, in step S2, as shown in FIG. 8, the ultrasound transducer unit 203 of the ultrasound endoscope 2 adjacent to an inner wall surface of the stomach 401, transmits and receives ultrasound using the ultrasound transducer unit 203, and confirms, with an ultrasound observation, that a luminal wall of a jejunum 402 is present on the outside of the stomach 401.

Moreover, using the ultrasound endoscope system 1, the surgeon confirms, as a safe area confirming step, in step S3, a pierceable area, which is a safe area without other luminal organs (including blood vessels and the like) 411, between the luminal wall of the stomach 401 and the luminal wall of the jejunum 402 in an observation range 410 of the ultrasound observation.

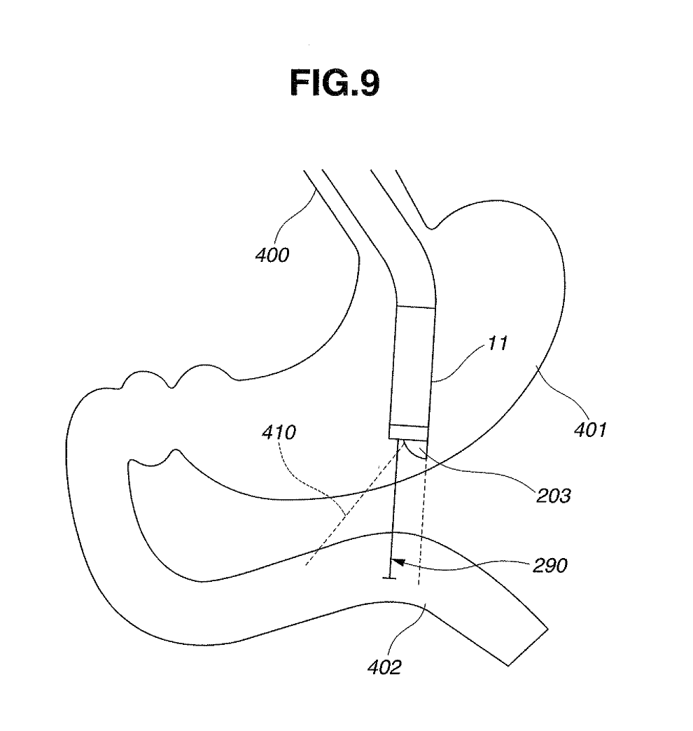

Using the ultrasound endoscope system 1, the surgeon pierces, as a capturing step, in step S4, a piercing treatment instrument 313 into the luminal wall of the stomach 401 and the luminal wall of the jejunum 402 through a first treatment instrument channel of the ultrasound endoscope 2 and inserts the G. W. with stopper 290 through a hollow path of the piercing treatment instrument 313. As shown in FIG. 9, using the ultrasound endoscope system 1, the surgeon retains the stopper 291 of the G. W. with stopper 290 from a distal end of the piercing treatment instrument 313 in the jejunum 402 and removes the piercing treatment instrument 313 from the treatment instrument channel of the ultrasound endoscope 2.

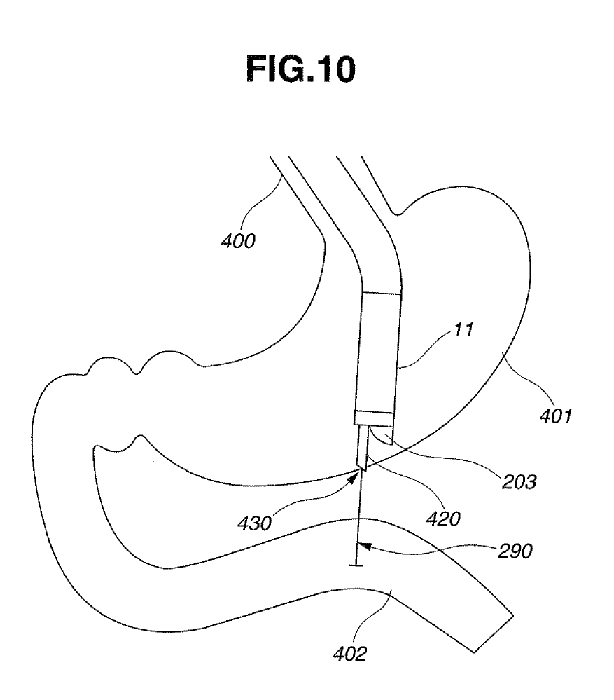

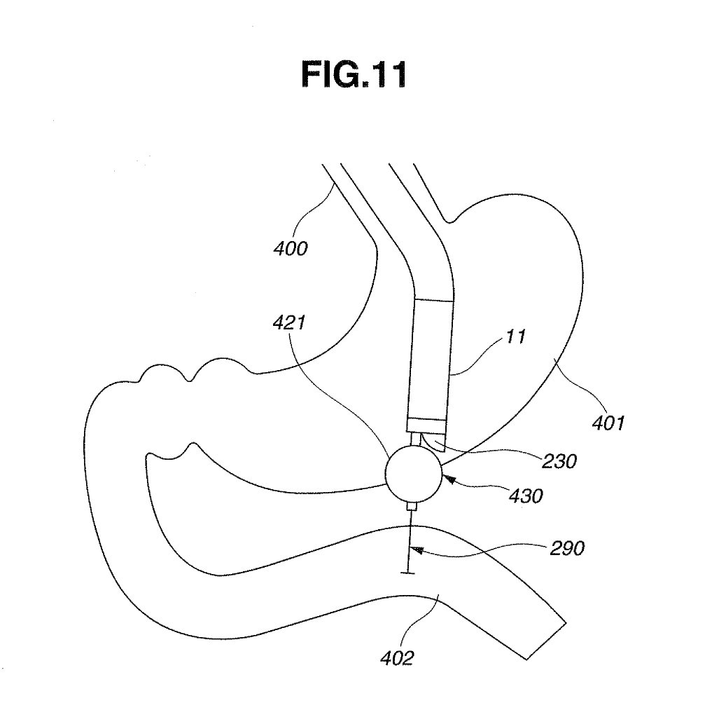

Subsequently, using the ultrasound endoscope system 1, the surgeon brings, as an opening portion forming step, in step S5, as shown in FIG. 10, instead of the piercing treatment instrument 313, a needle knife 420, through a hollow path of which the G. W. with stopper 290 is inserted, into contact with the luminal wall of the stomach 401 via the first treatment instrument channel of the ultrasound endoscope 2 to form an opening portion 430 in the luminal wall of the stomach 402. Using the ultrasound endoscope system 1, the surgeon pushes, as an opening portion expanding step, in step S6, as shown in FIG. 11, instead of the needle knife 420, a balloon dilator 421, through a hollow path of which the G. W. with stopper 290 is inserted, into the opening portion 430 via the first treatment instrument channel of the ultrasound endoscope 2 and inflates a balloon of the balloon dilator 421 to expand the opening portion 430.

The needle knife 420 has the hollow path for inserting the G. W. with stopper 290. However, the needle knife 420 is not limited to this and does not have to have the hollow path as long as the surgeon can insert the G. W. with stopper 290 as a guide after confirming safety with ultrasound scanning.

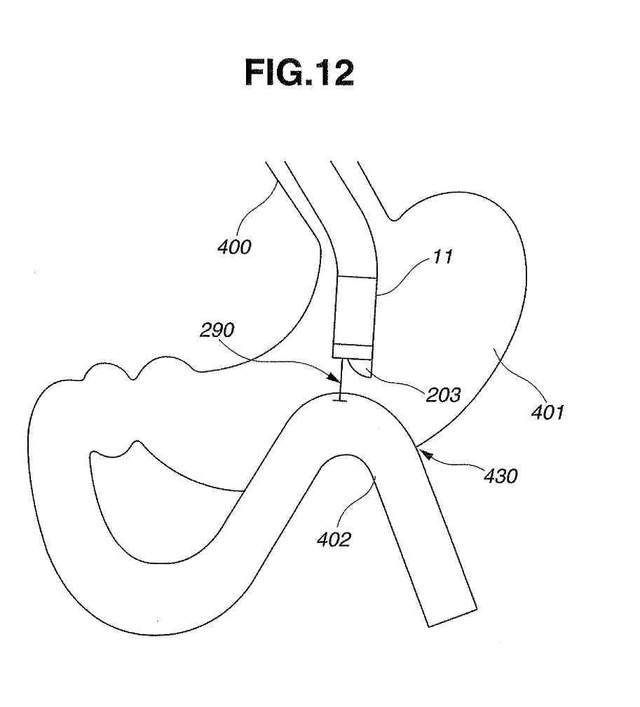

Next, using the ultrasound endoscope system 1, the surgeon draws out, as a pulling-in step, in step S7, as shown in FIG. 12, the wire portion 292 from the treatment instrument insertion openings 21 and pulls the G. W. with stopper 290 to thereby pull the jejunum 402 retained in the inside by the stopper 291 into the inside of the stomach 401 via the opening portion 430. In this step, in order to remove the G. W. with stopper 290, a caught portion of the stopper 291 is slightly dissected.

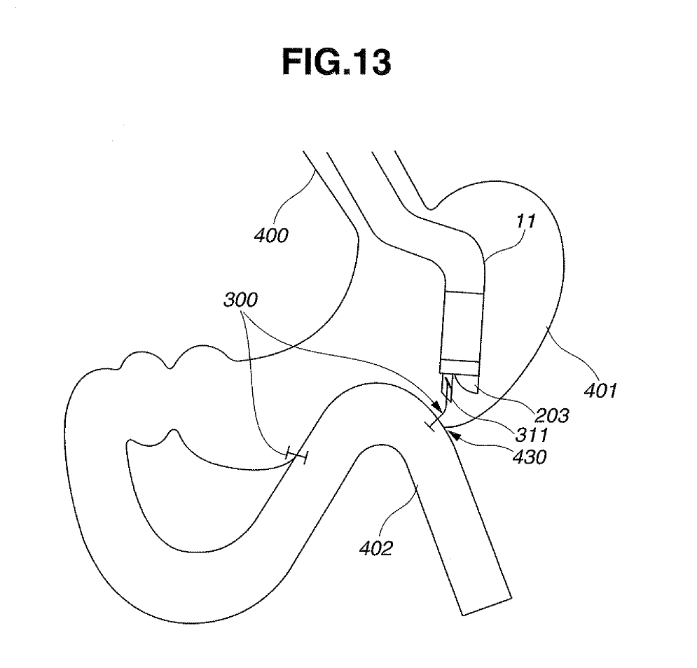

Subsequently, using the ultrasound endoscope system 1, the surgeon carries out, in treatment in a post step, a provisional fixing step for preventing the jejunum 402 from being drawn in. Using the ultrasound endoscope system 1, the surgeon holds, as the provisional fixing step, in step S8, as shown in FIG. 13, an organism tissue between the distal end bar members 301 and the center bar members 303 of the T-bar portions 300 using the T-bar portions 300 of the T-bar unit inserted through the first treatment instrument channel of the ultrasound endoscope 2 and using a grasping treatment instrument (not shown) inserted through the second treatment instrument channel of the ultrasound endoscope 2 and provisionally fixes an outer wail of the pulled-in jejunum 402 to the opening portion 430. The provisional fixing step may be carried out by using clips or the like instead of the T-bars.

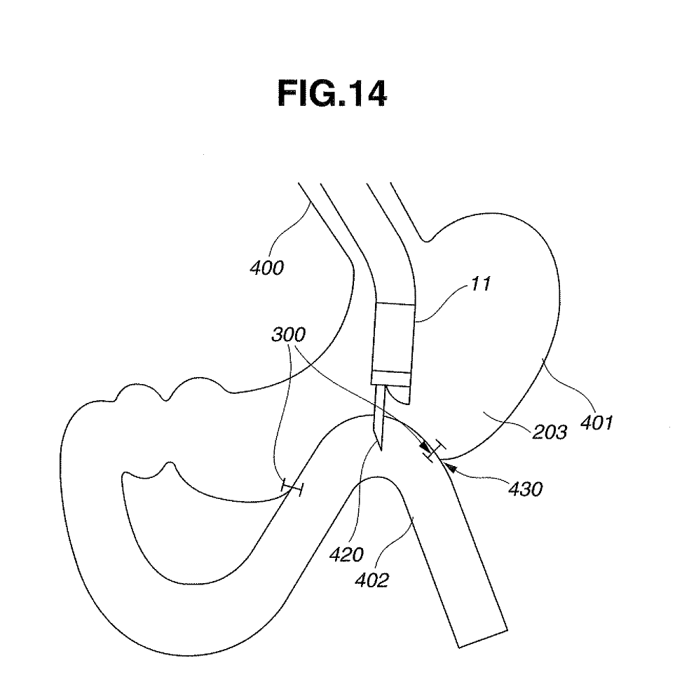

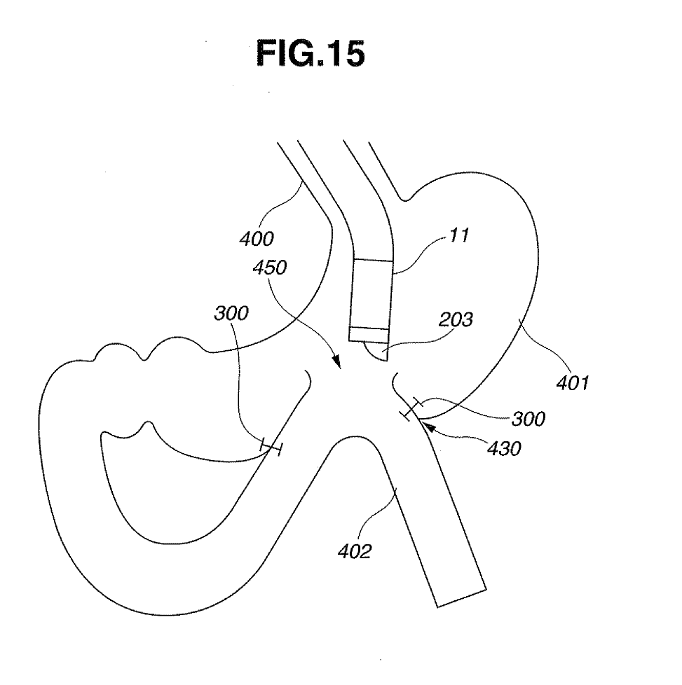

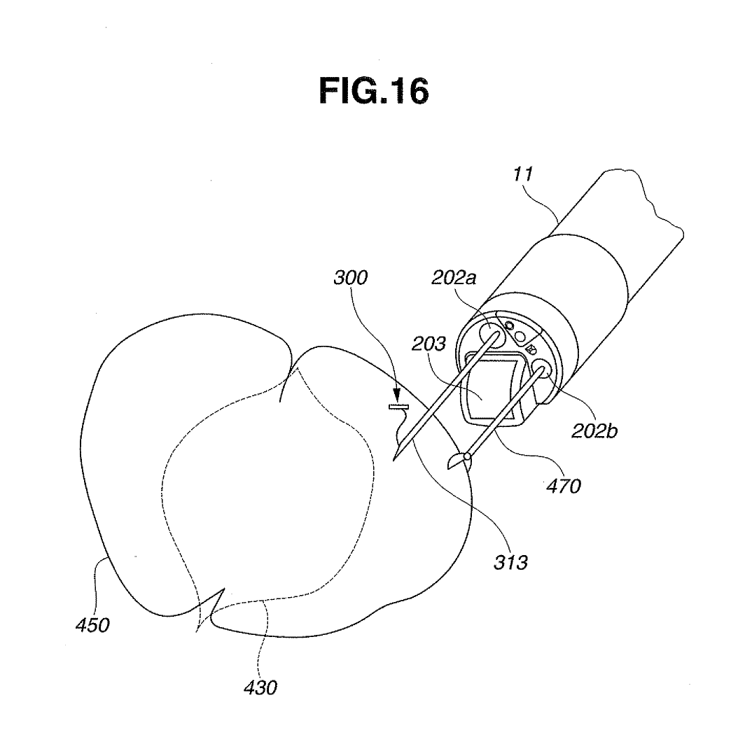

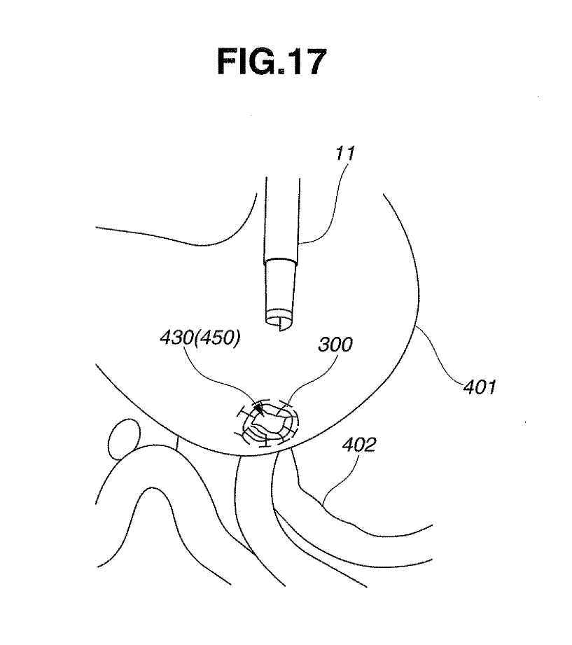

Using the ultrasound endoscope system 1, the surgeon inserts, as a treatment step, in step S9, as shown in FIG. 14, the needle knife 420 through the first treatment instrument channel of the ultrasound endoscope 2 again and, as shown in FIG. 15, forms an opening portion 450 in the outer wall of the pulled-in jejunum 850. Moreover, using the ultrasound endoscope system 1, the surgeon sutures, in step S10, as shown in FIGS. 16 and 17, the opening portion 450 of the jejunum 402 and the opening portion 430 of the stomach 401 using the T-bar portion 300 of the T-bar unit inserted through the first treatment instrument channel and using a grasping treatment instrument 470 inserted through the second treatment instrument channel and bypasses the jejunum 430 to the stomach 401. Using the ultrasound endoscope system 1, the surgeon removes, in step S11, the ultrasound endoscope 2 from the inside of the body cavity and finishes the processing.

As described above, according to the present embodiment, it is possible to obtain the following effects:
(1) since it is unnecessary to replace an endoscope, efficient manipulation can be carried out;
(2) since the surgeon can perform manipulation while checking the respective portions under the ultrasound observation, processing with less risk can be carried out; and
(3) since surgical manipulation is performed in a lumen, likelihood of complication can be reduced.

### Modification

A modification of the present embodiment is explained below. In the first embodiment described above, the bypass treatment of the stomach 401 and the jejunum 402 is explained as the example. In this modification, an example of lesion region excision treatment performed when a lesion region such as a tumor is present in the jejunum 402 is explained.

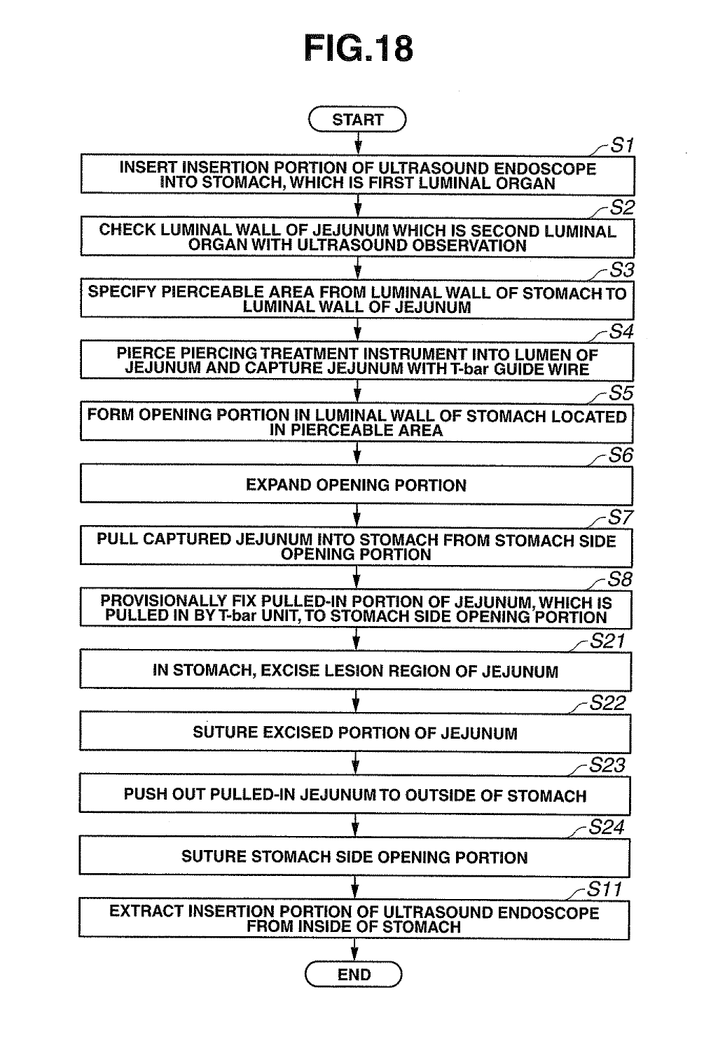

In this modification, instead of steps S9 and S10 explained with reference to FIG. 7, steps S21 to S24 are carried out as shown in FIG. 18.

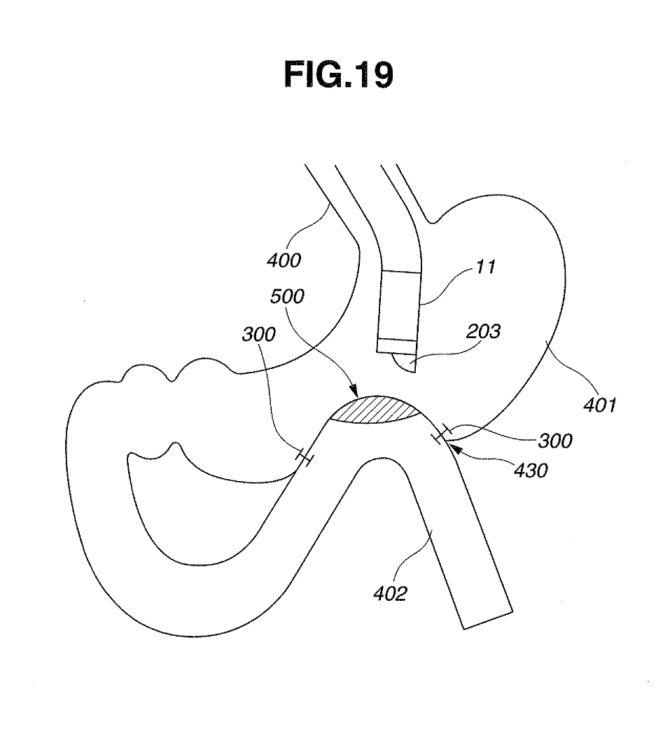

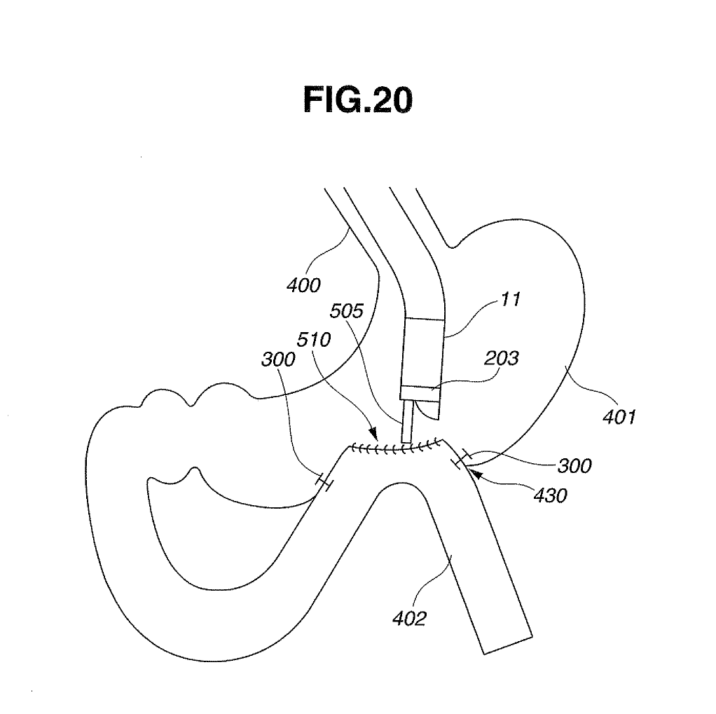

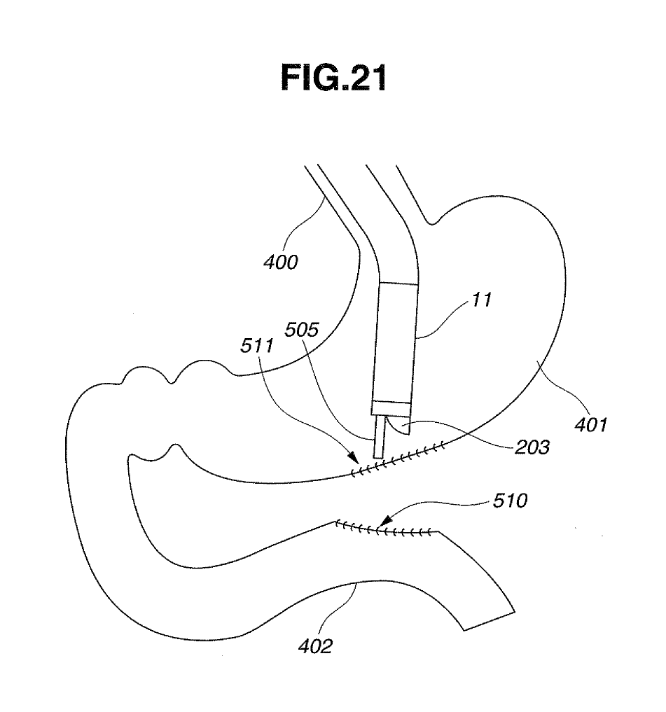

Using the ultrasound endoscope system 1, the surgeon specifies, after the step of step S8 explained with reference to FIG. 7, as a treatment step, in step S21, as shown in FIG. 19, a lesion region 500 of the jejunum 402 pulled into the inside of the stomach 401 and excises the lesion region 500 with the needle knife 420 (not shown). Using the ultrasound endoscope system 1, the surgeon sutures, in step S22, as shown in FIG. 20, an excision region 510 formed by excising the lesion region 500 using a suturing treatment instrument 505. Next, using the ultrasound endoscope system 1, the surgeon pushes out, in step S23, the pulled-in jejunum 402 to the outside of the stomach 401 from the opening portion 430. Using the ultrasound endoscope system 1, the surgeon sutures, in step S24, as shown in FIG. 21, the opening portion 430 using the suturing treatment instrument 505 and proceeds to step S11 explained with reference to FIG. 7.

In such a modification, it is possible to obtain effects same as those in the first embodiment.

In the present invention, it is evident that different embodiments can be implemented in a broad range on the basis of the present invention without departing from the spirit and the scope of the invention. The present invention is not limited by specific embodiments thereof except that the invention is limited by the appended claims.

What is claimed is:

1. An ultrasound endoscopic intraluminal organ treatment method comprising:
an endoscope inserting step of inserting an insertion portion of an ultrasound endoscope including one or more treatment instrument channels into a first object luminal organ from a natural orifice of a patient, the insertion portion having at a distal end thereof an optical observation portion for optically observing an observation region and an ultrasound observation portion that is capable of observing the observation region with ultrasound;
an adjacent organ confirming step of confirming a second object luminal organ adjacent to an outer wall surface of the first object luminal organ under an ultrasound observation of the ultrasound observation portion;
a capturing step of capturing the second object luminal organ under the ultrasound observation of the ultrasound observation portion;
an opening portion forming step of forming an opening portion in a luminal wall of the first object luminal organ;
a pulling-in step of pulling a part of the captured second object luminal organ into an inside of the first object luminal organ from the opening portion; and
a treatment step of applying treatment to the part of the second object luminal organ pulled into the inside of the first object luminal organ.

2. The ultrasound endoscopic intraluminal organ treatment method according to claim 1, further comprising:
a safe area confirming step of confirming, under the ultrasound observation of the ultrasound observation portion, a pierceable safe area, where there is no intra-body cavity organ other than the first object luminal organ and the second object luminal organ or no blood vessel, in an inter-organ area between the first object luminal organ and the second object luminal organ; and
a piercing step of piercing a piercing treatment instrument having a hollow path from an inner wall of the first object luminal organ into the second object luminal organ via the safe area, the piercing treatment instrument being inserted through at least one channel among plural treatment instrument channels, wherein
in the capturing step, the second object luminal organ is captured by inserting a guide wire including a stopper member at a distal end thereof into the hollow path of the piercing treatment instrument and retaining the stopper member in the second object luminal organ.

3. The ultrasound endoscopic intraluminal organ treatment method according to claim 2, wherein, in the opening portion forming step, the opening portion is formed in the luminal wall of the first object luminal organ by a dissecting treatment instrument having the guide wire as a guide.

4. The ultrasound endoscopic intraluminal organ treatment method according to claim 3, wherein the dissecting treatment instrument is a high-frequency treatment instrument having a hollow path through which the guide wire can be inserted.

5. The ultrasound endoscopic intraluminal organ treatment method according to claim 2, wherein, in the pulling-in step, a part of the captured second object luminal organ is pulled into the inside of the first object luminal organ by using the guide wire.

6. The ultrasound endoscopic intraluminal organ treatment method according to claim 1, further comprising an opening portion expanding step of expanding the opening portion, wherein
in the pulling-in step, a part of the captured second object luminal organ is pulled into the inside of the first object luminal organ from the expanded opening portion.

7. The ultrasound endoscopic intraluminal organ treatment method according to claim 2, further comprising an opening portion expanding step of expanding the opening portion, wherein
in the pulling-in step, a part of the captured second object luminal organ is pulled into the inside of the first object luminal organ from the expanded opening portion.

8. The ultrasound endoscopic intraluminal organ treatment method according to claim 1, further comprising a provisional fixing step of provisionally fixing the second object luminal organ, which is pulled into the inside of the first object luminal organ in the pulling-in step, to the opening portion.

9. The ultrasound endoscopic intraluminal organ treatment method according to claim 8, wherein, in the provisional fixing step, the second object luminal organ is provisionally fixed to the opening portion by a T-bar unit.

10. The ultrasound endoscopic intraluminal organ treatment method according to claim 1, wherein the treatment step is a bypass treatment step of applying bypass treatment for forming a second opening portion in a part of the second object luminal organ using plural treatment instruments inserted through the plural treatment instrument channels, suturing the opening portion and the second opening portion, and connecting the first object luminal organ and the second object luminal organ.

11. The ultrasound endoscopic intraluminal organ treatment method according to claim 10, wherein, in the bypass treatment step, the opening portion and the second opening portion are sutured by a T-bar unit.

12. The ultrasound endoscopic intraluminal organ treatment method according to claim 1, wherein
the part of the second object luminal organ pulled into the inside of the first object luminal organ in the pulling-in step is a region where a diseased part to be treated is present, and
the treatment step includes:
a diseased part excising step of excising the region where the diseased part is present, the diseased part excising step being carried out by plural treatment instruments inserted through the plural treatment instrument channels;
a first suturing step of suturing the excised region where the diseased part is present of the second object luminal organ;
a pushing-out step of pushing out the part of the second object luminal organ pulled in by the pulling-in step to an outside of the first object luminal organ from the opening portion; and
a second suturing step of suturing the opening portion after the pushing-out step.

13. The ultrasound endoscopic intraluminal organ treatment method according to claim 1, wherein an observation optical axis of the optical observation portion is substantially parallel to an insertion axis of the insertion portion, and at least one of the treatment instrument channels is provided in a position where a projecting direction of a treatment instrument projecting from a channel opening portion is in an observation range of the ultrasound observation portion.

14. An ultrasound endoscopic intraluminal organ treatment method comprising:
an endoscope inserting step of inserting an insertion portion of an ultrasound endoscope including plural treatment instrument channels into a first object luminal organ from a natural orifice of a patient under an optical observation of an optical observation portion for optically observing an observation region, the insertion portion having at a distal end thereof the optical observation portion and an ultrasound observation portion that is capable of observing the observation region with ultrasound;
an adjacent organ confirming step of confirming a second object luminal organ adjacent to an outer wall surface of the first object luminal organ under an ultrasound observation of the ultrasound observation portion;
a capturing step of capturing the second object luminal organ under the ultrasound observation of the ultrasound observation portion;
an opening portion forming step of forming an opening portion in a luminal wall of the first object luminal organ under the optical observation of the optical observation portion using a dissecting treatment instrument inserted through at least one of the plural treatment instrument channels;
a pulling-in step of pulling a part of the captured second object luminal organ into an inside of the first object luminal organ from the opening portion; and
a treatment step of applying, under the optical observation of the optical observation portion, predetermined treatment to the part of the second object luminal organ pulled into the inside of the first object luminal organ using plural treatment instruments inserted through the plural treatment instrument channels.

15. The ultrasound endoscopic intraluminal organ treatment method according to claim 14, further comprising:
a safe area confirming step of confirming, under the ultrasound observation of the ultrasound observation portion, a pierceable safe area, where there is no intra-body cavity organ other than the first object luminal organ and the second object luminal organ, in an inter-organ area between the first object luminal organ and the second object luminal organ; and
a piercing step of piercing a piercing treatment instrument having a hollow path from an inner wall of the first object luminal organ into the second object luminal organ via the safe area, the piercing treatment instrument being inserted through the treatment instrument channel, wherein
in the capturing step, the second object luminal organ is captured by inserting a guide wire including a stopper member at a distal end thereof into the hollow path of the piercing treatment instrument and retaining the stopper member in the second object luminal organ.

16. The ultrasound endoscopic intraluminal organ treatment method according to claim 14, further comprising an opening portion expanding step of expanding the opening portion, wherein
in the pulling-in step, a part of the captured second object luminal organ is pulled into the inside of the first object luminal organ from the expanded opening portion.

17. The ultrasound endoscopic intraluminal organ treatment method according to claim 14, further comprising a provisional fixing step of provisionally fixing the second object luminal organ, which is pulled into the inside of the first object luminal organ in the pulling-in step, to the opening portion.

18. The ultrasound endoscopic intraluminal organ treatment method according to claim 14, wherein the treatment step is a bypass treatment step of applying, under the optical observation of the optical observation portion, bypass treatment for forming a second opening portion in a part of the second object luminal organ using plural treatment instruments inserted through the plural treatment instrument channels, suturing the opening portion and the second opening portion, and connecting the first object luminal organ and the second object luminal organ.

19. The ultrasound endoscopic intraluminal organ treatment method according to claim 14, wherein
the part of the second object luminal organ pulled into the inside of the first object luminal organ in the pulling-in step is a region where a diseased part to be treated is present, and
the treatment step includes:
a diseased part excising step of excising the region where the diseased part is present, the diseased part excising step being carried out by plural treatment instruments inserted through the plural treatment instrument channels;
a first suturing step of suturing the excised region where the diseased part is present of the second object luminal organ;
a pushing-out step of pushing out the part of the second object luminal organ pulled in by the pulling-in step to an outside of the first object luminal organ from the opening portion; and
a second suturing step of suturing the opening portion after the pushing-out step.

20. The ultrasound endoscopic intraluminal organ treatment method according to claim 14, wherein an observation optical axis of the optical observation portion is substantially parallel to an insertion axis of the insertion portion, and at least one of the treatment instrument channels is provided in a position where a projecting direction of a treatment instrument projecting from a channel opening portion is in an observation range of the ultrasound observation portion.
